(19) United States
(12) Patent Application Publication
(10) Pub. No.: US 2023/0193346 A1
(43) Pub. Date: Jun. 22, 2023

(54) METHOD FOR DETECTING MYCOBACTERIUM TUBERCULOSIS

(71) Applicants: KOREA ATOMIC ENERGY RESEARCH INSTITUTE, Daejeon (KR); CHUNGNAM NATIONAL UNIVERSITY HOSPITAL, Daejeon (KR)

(72) Inventors: Jae Cheong LIM, Sejong-si (KR); Eun Ha CHO, Sejong-si (KR); So Young LEE, Daejeon (KR); Sung Hee JUNG, Gongju-si, Chungcheongnam-do (KR); Chae Uk CHUNG, Daejeon (KR)

(21) Appl. No.: 17/923,403

(22) PCT Filed: Apr. 29, 2021
(86) PCT No.: PCT/KR2021/005457

§ 371 Date: Nov. 4, 2022

(30) Foreign Application Priority Data
May 6, 2020 (KR) 10-2020-0054133

(51) Int. Cl. C12Q 1/16; C12Q 1/58

(52) U.S. Cl. CPC C12Q 1/16; C12Q 1/58; C12R 2001/32

(57) ABSTRACT

The present invention relates to a method for detecting Mycobacterium tuberculosis, which can replace the conventional culture method that takes a long time of four to eight weeks to detect active tuberculosis, and which is a method for detecting active Mycobacterium tuberculosis by using isotopes on a sample of a patient's sputum or bronchoalveolar lavage fluid.

### TECHNICAL FIELD

The present invention relates to a method for detecting Mycobacterium tuberculosis, which can replace the conventional culture method that takes a long time of four to eight weeks to detect active tuberculosis, and which is a method for detecting active Mycobacterium tuberculosis by using isotopes on a sample of a patient's sputum or bronchoalveolar lavage fluid.

### BACKGROUND ART

More than 10 million new cases of tuberculosis patients occur every year worldwide, and the number of deaths from tuberculosis has reached 1.8 million annually, which places a serious burden for the disease worldwide. In 2017, a total of 36,044 tuberculosis patient were diagnosed in Korea, which ranks first among the OECD countries and poses a great threat to pub health in Korea (KCDC, 2017) in addition, the tuberculosis problem in North Korea is the most serious in the world, and as of 2017, the number of tuberculosis patients is estimated be 130,000, and the death rate is also increasing significantly, and therefore, the tuberculosis problem between North Korea and South Korea is an important global issue (WHO, 2018).

Tuberculosis which is caused by Mycobacterium tuberculosis and the like is classified into latent tuberculosis and active tuberculosis according to the condition of Mycobacterium tuberculosis The condition in which Mycobacterium tuberculosis is dormant and not contagious is called latent tuberculosis, and the condition in which tuberculosis symptoms and contagiousness occur due to various causes such as reduced immunity is called active tuberculosis.

In order to diagnose active tuberculosis, it is necessary to detect live Mycobacterium tuberculosis in samples generated from patients, such as sputum, bronchoalveolar lavage fluid and the like. Currently, the culture method for culturing and confirming live Mycobacterium tuberculosis from a patient's sputum sample is used as a gold standard. However, in order to detect Mycobacterium tuberculosis by the culture method, a long time of 4 to 8 weeks is required. Since the time required for the diagnosis of active tuberculosis is long, it is difficult to treat tuberculosis in an early stage, and since isolation measures are difficult, it becomes a major cause of tuberculosis transmission. Therefore, there is a need for a new technique for diagnosing active tuberculosis by more rapidly detecting live Mycobacterium tuberculosis in a patient's sample.

Accordingly, the inventors of the present invention researched a method for diagnosing Mycobacterium tuberculosis on site, and as a result of pre-treating a sample of sputum or bronchoalveolar lavage from a patient suspected of tuberculosis and treating urea (′³C-urea) composed of a carbon isotope, the present invention was completed by confirming that rapid detection is possible within several hours or a week and the accuracy is increased.

### DISCLOSURE

### Technical Problem

The present invention relates to a method for detecting Mycobacterium tuberculosis, which can replace the conventional culture method that takes a long time of four to eight weeks to detect active tuberculosis, and which is a method for detecting active Mycobacterium tuberculosis by using isotopes on a sample of a patient's sputum or bronchoalveolar lavage fluid.

However, the technical problems to be achieved by the present invention is not limited to the above-mentioned problem, and other problems that are not mentioned will be clearly understood by those skilled in the art from the following description.

### Technical Solution

In order to achieve the above objects, the present invention provides a composition for detecting Mycobacterium tuberculosis in sputum or bronchoalveolar lavage fluid, including urea (¹³C-urea or ¹⁴C-urea) composed of a carbon isotope.

In addition, the present invention provides a method for detecting Mycobacterium tuberculosis, including the steps of (a) treating a biological sample isolated from a patient suspected of tuberculosis with NaOH or N-acetyl-L-cysteine (NALC)-NaOH; (b) culturing the sample treated with NaOH or N-acetyl-L-cysteine (NALC)-NaOH in a liquid medium for culturing Mycobacterium tuberculosis; (c) treating a culture product cultured in the liquid medium with urea (¹³C-urea or ¹⁴C-urea) composed of a carbon isotope; and (d) measuring ¹³CO₂ or ¹⁴CO₂ of the culture product.

In addition, the present invention provides a method for providing information for diagnosing tuberculosis, including the steps of (a) treating a biological sample isolated from a patient suspected of tuberculosis with NaOH or N-acetyl-L-cysteine (NALC)-NaOH; (b) culturing the sample treated with NaOH or N-acetyl-L-cysteine (NALC)-NaOH in a liquid medium for culturing Mycobacterium tuberculosis; (c) treating a culture product cultured in the liquid medium with urea (¹³C-urea or ¹⁴C-urea) composed of a carbon isotope; (d) measuring ¹³CO₂ or ¹⁴CO₂ of the culture product; and (e) determining the patient as a tuberculosis patient if the level of ¹³CO₂ or ¹⁴CO₂ is higher than a normal sample.

### Advantageous Effects

The method for detecting Mycobacterium tuberculosis according to the present invention treats urea (¹³C-urea or ¹⁴C-urea) composed of a carbon isotope in a patient's sputum or bronchoalveolar lavage fluid such that there is an effect that active tuberculosis can be diagnosed within a few hours or 1 week. In addition, by detecting active Mycobacterium tuberculosis in sputum and bronchoalveolar lavage fluid, it is possible to increase the detection accuracy, and thus, there is an effect of on-site diagnosis of active tuberculosis by the method of the present invention using the same.

### DESCRIPTION OF DRAWINGS

FIG. 1 is a mimetic diagram of the Mycobacterium tuberculosis detection process of the present invention.

FIG. 2 is the results of treating sputum without Mycobacterium tuberculosis (TB-, Control) and sputum of tuberculosis patients (TB+, Test) in the same way, and measuring changes in the concentration of ¹³CO₂ with an isotope ratio mass spectrometer (Gasbench II & Thermo Fisher Delta V advantage IRMS) within 1 day.

### BEST MODE

As a result of researching a method for diagnosing Mycobacterium tuberculosis on site, the inventors of the present invention completed the present invention by confirming that active Mycobacterium tuberculosis can be detected by using isotopes in a patient's sputum or bronchoalveolar lavage sample.

The present invention provides a composition for detecting Mycobacterium tuberculosis in sputum or bronchoalveolar lavage fluid, including urea (¹³C-urea or ¹⁴C-urea) composed of a carbon isotope.

As used herein, the term “tuberculosis” is a disease caused by Mycobacterium tuberculosis or the like, and it is classified into latent tuberculosis and active tuberculosis according to the status of Mycobacterium tuberculosis. The term “active tuberculosis” refers to a state in which tuberculosis symptoms and contagiousness are caused due to various causes such as reduced immunity or the like. Prompt diagnosis of active tuberculosis is of utmost importance in order to suppress the transmission of tuberculosis and to treat the same at an early stage. Currently, the culture method for culturing and confirming live Mycobacterium tuberculosis from a patient's sputum sample is used as a gold standard. However, in order to detect Mycobacterium tuberculosis by the culture method, a long time of 4 to 8 weeks is required, and in the case of a breath test using the patient's exhaled breath by using ¹³C-urea or ¹⁴C-urea, since most bacteria have the urease enzyme, it is possible to diagnose other bacteria besides Mycobacterium tuberculosis, which has a disadvantage in that the accuracy of diagnosis is sharply reduced. For example, the breath test method using ¹³C-urea or ¹⁴C-urea is also used to detect infected Helicobacter pylori.

Accordingly, the inventors of the present invention researched a method for diagnosing Mycobacterium tuberculosis with high accuracy on site, and as a result of pre-treating the sputum or bronchoalveolar lavage fluid of a patient suspected of tuberculosis, and then treating the urea composed of a carbon isotope, it was confirmed that it is possible to detect Mycobacterium tuberculosis quickly within several hours or 1 week and the accuracy is increased (Table 2 and Table 3).

In an exemplary embodiment of the present invention, the Mycobacterium tuberculosis may be active Mycobacterium tuberculosis.

In an exemplary embodiment of the present invention, the composition may detect a culture product for 1 to 7 days as a target.

In addition, the present invention provides a method for detecting Mycobacterium tuberculosis, including the steps of (a) treating a biological sample isolated from a patient suspected of tuberculosis with NaOH or N-acetyl-L-cysteine (NALC)-NaOH; (b) culturing the sample treated with NaOH or N-acetyl-L-cysteine (NALC)-NaOH in a liquid medium for culturing Mycobacterium tuberculosis; (c) treating a culture product cultured in the liquid medium with urea (¹³C-urea or ¹⁴C-urea) composed of a carbon isotope; and (d) measuring ¹³CO₂ or ¹⁴CO₂ of the culture product.

In an exemplary embodiment of the present invention, the biological sample may be sputum or bronchoalveolar lavage fluid.

In an exemplary embodiment of the present invention, the pH of the culture cultured in the liquid medium in step (c) may be pH 5 to pH 8, but the present invention is not limited thereto.

In addition, the present invention provides a method for providing information for diagnosing tuberculosis, including the steps of (a) treating a biological sample isolated from a patient suspected of tuberculosis with NaOH or N-acetyl-L-cysteine (NALC)-NaOH; (b) culturing the sample treated with NaOH or N-acetyl-L-cysteine (NALC)-NaOH in a liquid medium for culturing Mycobacterium tuberculosis; (c) treating a culture product cultured in the liquid medium with urea (¹³C-urea or ¹⁴C-urea) composed of a carbon isotope; (d) measuring ¹³CO₂ or ¹⁴CO₂ of the culture product; and (e) determining the patient as a tuberculosis patient if the level of ¹³CO₂ or ¹⁴CO₂ is higher than a normal sample.

In an exemplary embodiment of the present invention, the biological sample may be sputum or bronchoalveolar lavage fluid.

### Modes of the Invention

Hereinafter, preferred examples are presented to help the understanding of the present invention. However, the following examples are only provided for easier understanding of the present invention, and the contents of the present invention are not limited by the following examples.

### Example 1. Detection of Mycobacterium tuberculosis

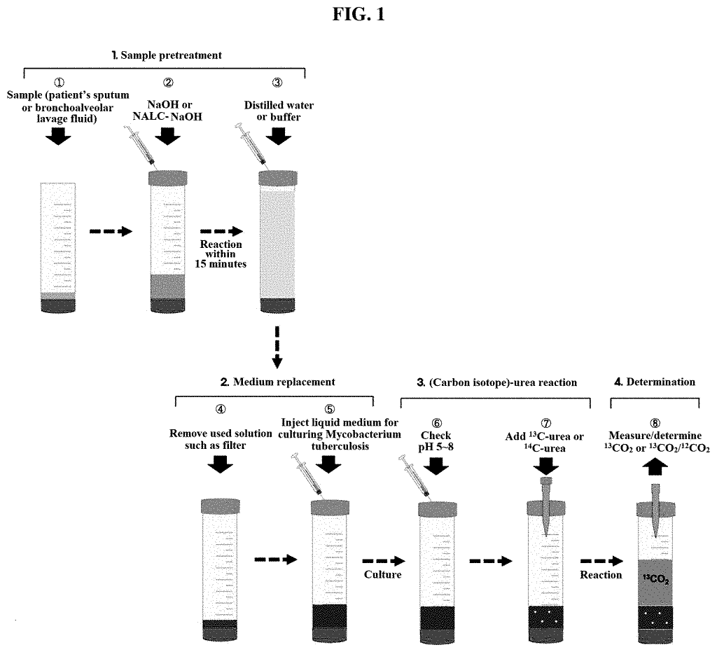

As with the standard method (culture method) used for the existing diagnosis of active tuberculosis, sputum or bronchoalveolar lavage from tuberculosis patients was used as diagnostic samples. The used samples were confirmed to contain Mycobacterium tuberculosis by the standard method (culture method) (Tuberc. Respir. Dis. 2015; 78: 64-71; Yon Ju Ryu, M.D.). As shown in FIG. 1, the patient's sample was collected in a sealable container. 5 to 10 mL of NaOH solution or N-acetyl-L-cysteine (NALC)-NaOH (KS Seongnam, Korea) was injected into the container, mixed and reacted at room temperature for 15 minutes. 40 to 50 mL of distilled water or buffer was injected and mixed to neutralize the solution. Through this process, all other bacteria except for Mycobacterium tuberculosis were killed, leaving only live Mycobacterium tuberculosis. For the smooth metabolic activity and culture of Mycobacterium tuberculosis, the solution was replaced with a liquid medium for tuberculosis culture. In order to prevent the loss of Mycobacterium tuberculosis in the sample, it was purified by using a filter membrane having a pore size of 1 μm or less, and recovered as 5 to 10 mL of a liquid medium for culturing Mycobacterium tuberculosis. Depending on the condition of the sample, the next detection step was performed immediately or after culturing within 1 week. Various samples were evaluated as shown in Table 1 below.

TABLE 1

| Classification | Sample name | Sample content |
|---|---|---|
| Control group | 1 | control (TB − sputum) |
| | 2 | control (TB − sputum) |
| | 3 | control (TB − sputum) |
| | 4 | control (TB − sputum) |
| Experimental | 5 | TB + sputum |
| group | 6 | TB + sputum |
| | 7 | TB + sputum |
| | 8 | TB + sputum |
| Control group | 9 | control (TB − bronchoalveolar |
| | | lavage fluid) |
| | 10 | control (TB − bronchoalveolar |
| | | lavage fluid) |
| | 11 | control (TB − bronchoalveolar |
| | | lavage fluid) |
| | 12 | control (TB − bronchoalveolar |
| | | lavage fluid) |
| Experimental | 13 | TB + bronchoalveolar lavage |
| group | | fluid |
| | 14 | TB + bronchoalveolar lavage |
| | | fluid |
| | 15 | TB + bronchoalveolar lavage |
| | | fluid |
| | 16 | TB + bronchoalveolar lavage |
| | | fluid |

¹³C-urea was added to the samples in Table 1 and reacted within 1 hour. In contrast to the control group (sputum bronchoalveolar lavage sample which was not infected with Mycobacterium tuberculosis), the concentration of ¹³CO₂ generated in the container was evaluated by using mass spectrometry or infrared spectroscopy. If an increase in the ¹³CO₂ concentration or an increase in the ¹³CO₂/¹²CO₂ value was confirmed, the presence of live Mycobacterium tuberculosis in the patient's sample was diagnosed as active tuberculosis. When the ¹³CO₂ concentration of the experimental group compared to the control group was measured by using the carbon dioxide carbon isotope ratio analyzer (POCone, Otsuka electronics Co.) equipment used in the existing breath test method, it was confirmed that the ¹³CO₂ level increased in all the experimental groups such that live Mycobacterium tuberculosis was detected in the samples (Table 2).

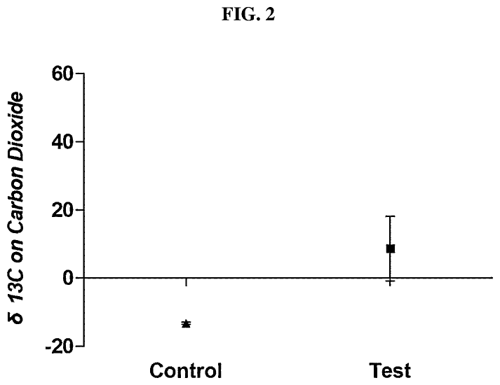

In addition, sputum without Mycobacterium tuberculosis (TB-, Control) and sputum of tuberculosis patients (TB+, Test) were treated in the same way, and changes in the concentration of ¹³CO₂ were measured by using an isotope ratio mass spectrometer (Gasbench II & Thermo Fisher Delta V advantage IRMS) within 1 day. As a result, significant increases in the ¹³CO₂ concentration were confirmed in the sputum samples of tuberculosis patients, which confirmed that Mycobacterium tuberculosis could be detected (FIG. 2). The measured values were derived as relative values in accordance with the Vienna Pee Dee Belemnite (VPDB) international standards.

This indicates that when Mycobacterium tuberculosis is detected by using the conventional culture method, it takes about 4 weeks, but the method according to the present invention takes only 1 day to 1 week.

TABLE 2

| Name of measurement sample | | ¹³CO₂ change rate | |
|---|---|---|---|
| Sample No. 1 | Sample No. 2 | (Sample No. 2 | |
| (control | (experimental | ¹³CO₂/Sample | Measurement |
| group) | group) | No. 1 ¹³CO₂) | results |
| 1 | 2 | 0.0 | TB − (negative) |
| 1 | 5 | 1.7 | TB + (positive) |
| 2 | 6 | 2.0 | TB + (positive) |
| 3 | 7 | 2.2 | TB + (positive) |
| 4 | 8 | 1.0 | TB + (positive) |
| 9 | 13 | 1.9 | TB + (positive) |
| 10 | 14 | 0.9 | TB + (positive) |
| 11 | 15 | 2.4 | TB + (positive) |
| 12 | 16 | 3.9 | TB + (positive) |

The above description of the present invention is for illustration, and those of ordinary skill in the art to which the present invention pertains will understand that it may be easily modified into other specific forms without changing the technical spirit or essential features of the present invention. Therefore, it should be understood that the exemplary embodiments described above are illustrative in all respects and not restrictive.

1. A composition for detecting Mycobacterium tuberculosis in sputum or bronchoalveolar lavage fluid, comprising urea (¹³C-urea or ¹⁴C-urea) composed of a carbon isotope.

2. The composition of claim 1, wherein the Mycobacterium tuberculosis is active Mycobacterium tuberculosis.

3. The composition of claim 1, wherein the composition detects a culture product for 1 to 7 days as a target.

4. A method for detecting Mycobacterium tuberculosis, comprising the steps of:
(a) treating a biological sample isolated from a patient suspected of tuberculosis with NaOH or N-acetyl-L-cysteine (NALC)-NaOH;
(b) culturing the sample treated with NaOH or N-acetyl-L-cysteine (NALC)-NaOH in a liquid medium for culturing Mycobacterium tuberculosis;
(c) treating a culture product cultured in the liquid medium with urea (¹³C-urea or ¹⁴C-urea) composed of a carbon isotope; and
(d) measuring ¹³CO₂ or ¹⁴CO₂ of the culture product.

5. The method of claim 4, wherein the biological sample is sputum or bronchoalveolar lavage fluid.

6. The method of claim 4, wherein the pH of the culture product cultured in the liquid medium of step (c) is pH 5 to pH 8.

7. A method for providing information for diagnosing tuberculosis, comprising the steps of:
(a) treating a biological sample isolated from a patient suspected of tuberculosis with NaOH or N-acetyl-L-cysteine (NALC)-NaOH;
(b) culturing the sample treated with NaOH or N-acetyl-L-cysteine (NALC)-NaOH in a liquid medium for culturing Mycobacterium tuberculosis;
(c) treating a culture product cultured in the liquid medium with urea (¹³C-urea or ¹⁴C-urea) composed of a carbon isotope;
(d) measuring ¹³CO₂ or ¹⁴CO₂ of the culture product; and
(e) determining the patient as a tuberculosis patient if the level of ¹³CO₂ or ¹⁴CO₂ is higher than a normal sample.

8. The method of claim 7, wherein the biological sample is sputum or bronchoalveolar lavage fluid.
